(19) United States
(12) Patent Application Publication
(10) Pub. No.: US 2006/0188441 A1
(43) Pub. Date: Aug. 24, 2006

(54) Radiolabeled gallium complexes, methods for synthesis and use for PET imaging of EGFR expression in malignant tumors

(76) Inventors: Vladimir Tolmachev, Uppsala (SE); Bengt Langstrom, Uppsala (SE); Asa Liljegren Sundberg, Uppsala (SE); Irina Velikyan, Uppsala (SE)

Correspondence Address: Amersham Health, Inc;IP Department, 101 Carnegie Center, Princeton, NJ, 08540, US

(21) Appl. No.: 11/358,681

(22) Filed: Feb. 21, 2006

Related U.S. Application Data
(60) Provisional application No. 60/655,139, filed on Feb. 22, 2005.

(51) Int. Cl. A61K 51/00; C07K 14/475

(52) U.S. Cl. 424/1.69; 530/399

(57) ABSTRACT

Methods for labeling synthesis of radiolabeled gallium complex by microwave activation are provided. The resultant gallium-isotope labeled compounds are useful as radiopharmaceuticals, especially for use in Positron Emission Tomography (PET). A method for imaging EGFR overexpression in tumors using PET is also provided.

### FIELD OF THE INVENTION

The present invention relates to radiolabeled gallium complexes and methods of synthesis thereof. The radiolabeled gallium complexes according to the present invention are useful as radiopharmaceuticals, specifically for use in Positron Emission Tomography (PET). They are particularly useful for the detection of epidermal growth factor receptor (EGFR) expression in malignant tumors.

### BACKGROUND OF THE INVENTION

The epidermal growth factor receptor (EGFR), also known as HER1 and ErbB-1, is a transmembrane protein belonging to the tyrosine kinase receptor family. Activation of EGFR causes signaling leading to cell division, increasing motility and suppression of apoptosis (Yarden Y, Sliwkowski M X. Untangling the ErbB signaling network. Nat Rev Mol Cell Biol. 2001; 2(2): 127-137). In a number of carcinomas, amplification or translocation of EGFR genes causes an increased transcription and a subsequent high level of EGFR expression (Collins V P. Amplified genes in human gliomas. Semin Cancer Biol. 1993; 4(1): 27-32; Bigner S H, Burger P C, Wong A J, et al. Gene amplification in malignant human gliomas: clinical and histopathologic aspects. J Neuropathol Exp Neurol. 1988; 47(3): 191-205). Overexpression of EGFR is documented in e.g. carcinomas of breast (Walker R A, Dearing S J. Expression of epidermal growth factor receptor mRNA and protein in primary breast carcinomas. Breast Cancer Res Treat. 1999; 53(2): 167-176; Witton C J, Reeves J R, Going J J, Cooke T G, Bartlett J M. Expression of the HER1-4 family of receptor tyrosine kinases in breast cancer. J Pathol. 2003; 200(3): 290-297), lung (Hirsch F R, Varella-Garcia M, Bunn P A, Jr., et al. Epidermal growth factor receptor in non-small-cell lung carcinomas: correlation between gene copy number and protein expression and impact on prognosis. J Clin Oncol. 2003; 21(20): 3798-3807) and urinary bladder (Neal D E, Mellon K. Epidermal growth factor receptor and bladder cancer: a review. Urol Int. 1992; 48(4): 365-371). A high level of EGFR expression could provide malignant cells with an advantage in survival by increasing cell proliferation and metastatic spread, and a decreasing apoptosis. For the moment, a number of approaches to suppress tumor growth by inactivation of EGFR signaling are in clinical use or under active evaluation. These approaches are based either on blocking ligand binding to the EGFR extracellular domain using anti-EGFR antibodies or preventing intracellular signaling with selective tyrosine kinase inhibitors (Castillo L, Etienne-Grimaldi M C, Fischel J L, Formento P, Magne N, Milano G. Pharmacological background of EGFR targeting. Ann Oncol. 2004; 15(7): 1007-1012).

Detection of EGFR expression in tumors has documented prognostic and predictive values. It was shown that overexpression of EGFR is associated with poor survival and recurrences in colon (Resnick M B, Routhier J, Konkin T, Sabo E, Pricolo V E. Epidermal growth factor receptor, c-MET, beta-catenin, and p53 expression as prognostic indicators in stage II colon cancer: a tissue microarray study. Clin Cancer Res. 2004; 10(9): 3069-3075), rectal (Kopp R, Rothbauer E, Mueller E, Schildberg F W, Jauch K W, Pfeiffer A. Reduced survival of rectal cancer patients with increased tumor epidermal growth factor receptor levels. Dis Colon Rectum. 2003; 46(10): 1391-1399), non-small-cell lung (Selvaggi G, Novello S, Torri V, et al. Epidermal growth factor receptor overexpression correlates with a poor prognosis in completely resected non-small-cell lung cancer. Ann Oncol. 2004; 15(1): 28-32) and breast cancer (Witton C J, Reeves J R, Going J J, Cooke T G, Bartlett J M. Expression of the HER1-4 family of receptor tyrosine kinases in breast cancer. J Pathol. 2003; 200(3): 290-297; Tsutsui S, Kataoka A, Ohno S, Murakami S, Kinoshita J, Hachitanda Y. Prognostic and predictive value of epidermal growth factor receptor in recurrent breast cancer. Clin Cancer Res. 2002; 8(11): 3454-3460). It was suggested that EGFR expression status could identify a subgroup of patients within advanced nasopharyngeal carcinoma that will have a poor outcome after induction chemotherapy and radiotherapy (Chua D T, Nicholls J M, Sham J S, Au G K. Prognostic value of epidermal growth factor receptor expression in patients with advanced stage nasopharyngeal carcinoma treated with induction chemotherapy and radiotherapy. Int J Radiat Oncol Biol Phys. 2004; 59(1): 11-20). There are evidences that expression of EGFR correlates with disease relapse and progression to androgen-independence in prostate cancer (Di Lorenzo G, Tortora G, D'Armiento F P, et al. Expression of epidermal growth factor receptor correlates with disease relapse and progression to androgen-independence in human prostate cancer. Clin Cancer Res. 2002; 8(11): 3438-3444). Apparently, detection of EGFR in clinical practice might influence patient management including questions of relevance of the use of EGFR-targeted drugs.

Detection of EGFR is possible in surgical samples or samples of fine-needle biopsies using immunohistochemistry or FISH technique. However, nuclear medicine visualization may provide advantages due to evaluation of the whole volume of both the primary tumor and metastases, and enabling to avoid false-negative results associated with sampling errors and heterogeneity of EGFR expression.

Indium-111 labelled anti-EGFR antibody 425 was successfully used for detection of malignant gliomas (Dadparvar S, Krishna L, Miyamoto C, et al. Indium-111-labeled anti-EGFr-425 scintigraphy in the detection of malignant gliomas. Cancer. 1994; 73(3 Suppl): 884-889). Tc-99m-labellled anti-EGFR humanized antibodies hR3 and C225 are under clinical evaluation (Vallis K A, Reilly R M, Chen P, et al. A phase I study of 99 mTc-hR3 (DiaCIM), a humanized immunoconjugate directed towards the epidermal growth factor receptor. Nucl Med Commun. 2002; 23(12): 1155-1164; Schechter N R, Wendt R E, 3rd, Yang D J, et al. Radiation dosimetry of 99 mTc-labeled C225 in patients with squamous cell carcinoma of the head and neck. J Nucl Med. 2004; 45(10): 1683-1687). It should be noted, however, that the use of bulky antibody proteins might complicate radioconjugate diffusion through healthy tissues and into tumor. An alternative to anti-EGFR antibodies might be the use of a natural ligand, epidermal growth factor (EGF) as a targeting vector for delivery of radionuclides to tumor cells (Schechter N R, Wendt R E, 3rd, Yang D J, et al. Radiation dosimetry of 99mTc-labeled C225 in patients with squamous cell carcinoma of the head and neck. J Nucl Med. 2004; 45(10): 1683-1687). The small molecular weight of EGF, 6.2 kDa, might enable fast tumor penetration and fast blood clearance, providing good contrast of the image. Earlier, ¹³¹I-labellled EGF has been successfully used for visualization of lung cancer (Cuartero-Plaza A, Martinez-Miralles E, Rosell R, Vadell-Nadal C, Farre M, Real F X. Radiolocalization of squamous lung carcinoma with 131I-labeled epidermal growth factor. Clin Cancer Res. 1996; 2(1): 13-20). However, poor cellular retention of radiohalogens might lead to decreased tumor accumulation and suboptimal imaging contrast, and the use of radiometals might be a better choice for labeling of EGF (Orlova A, Bruskin A, Sjostrom A, Lundqvist H, Gedda L, Tolmachev V. Cellular processing of (125)I- and (111)in-labeled epidermal growth factor (EGF) bound to cultured A431 tumor cells. Nucl Med Biol. 2000; 27(8): 827-835). Different single-photon radiometal labels for EGF have been proposed. ¹¹¹In (T_{1/2}=2,8 d) has been attached to EGF using the monoamide DTPA (Orlova A, Bruskin A, Sjostrom A, Lundqvist H, Gedda L, Tolmachev V. Cellular processing of (125)I- and (111)in-labeled epidermal growth factor (EGF) bound to cultured A431 tumor cells. Nucl Med Biol. 2000; 27(8): 827-835; Reilly R M, Gariepy J. Factors influencing the sensitivity of tumor imaging with a receptor-binding radiopharmaceutical. J Nucl Med. 1998; 39(6): 1036-1043) or isothiocyanate-benzyl-DTPA (Sundberg A L, Orlova A, Bruskin A, et al. [(111)In]Bz-DTPA-hEGF: Preparation and in vitro characterization of a potential anti-glioblastoma targeting agent. Cancer Biother Radiopharm. 2003; 18(4): 643-654). MAG3 (Hnatowich D J, Qu T, Chang F, Ley A C, Ladner R C, Rusckowski M. Labeling peptides with technetium-99m using a bifunctional chelator of a N-hydroxysuccinimide ester of mercaptoacetyltriglycine. J Nucl Med. 1998; 39(1): 56-64), introduced SH-group (Capala J, Barth R F, Bailey M Q, Fenstermaker R A, Marek M J, Rhodes B A. Radiolabeling of epidermal growth factor with 99 mTc and in vivo localization following intracerebral injection into normal and glioma-bearing rats. Bioconjug Chem. 1997; 8(3): 289-295) or HYNIC (Tolmachev, unpublished data) have been applied for labeling of EGF with generator-produced ^{99m}Tc (T_{1/2}=6 h). It may be of advantage, however, to use a positron-emitting label for EGF, since positron emission tomography (PET), compared to SPECT, is a superior detection technique in sensitivity, resolution, and quantification (Lundqvist H, Lubberink M, Tolmachev V. Positron Emission Tomography. European Journal of Physics. 1999; 19: 537-552; Lundqvist H, Tolmachev V. Targeting peptides and positron emission tomography. Biopolymers. 2002; 66(6): 381-392).

PET imaging is a tomographic nuclear imaging technique that uses radioactive tracer molecules that emit positrons. When a positron meets an electron, the both are annihilated and the result is a release of energy in form of gamma rays, which are detected by the PET scanner. By employing natural substances that are used by the body as tracer molecules, PET does not only provide information about structures in the body but also information about the physiological function of the body or certain areas therein. A common tracer molecule is for instance 2-fluoro-2-deoxy-D-glucose (FDG), which is similar to naturally occurring glucose, with the addition of an ¹⁸F-atom. Gamma radiation produced from said positron-emitting fluorine is detected by the PET scanner and shows the metabolism of FDG in certain areas or tissues of the body, e.g. in the brain or the heart. The choice of tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule.

Commonly used radionuclides in PET tracers are ¹¹C, ¹⁸F, ¹⁵O ¹³ N or ⁷⁶Br. Recently, new PET tracers were produced that are based on radiolabelled metal complexes comprising a bifunctional chelating agent and a radiometal. Bifunctional chelating agents are chelating agents that coordinate to a metal ion and are linked to a targeting vector that will bind to a target site in the patient's body. Such a targeting vector may be a peptide that binds to a certain receptor, probably associated with a certain area in the body or with a certain disease. A targeting vector may also be an oligonucleotide specific for e.g. an activated oncogene and thus aimed for tumour localisation. The advantage of such complexes is that the bifunctional chelating agents may be labelled with a variety of radiometals like, for instance, ⁶⁸Ga, ²¹³Bi or ⁸⁶Y. In this way, radiolabelled complexes with special properties may be “tailored” for certain applications.

⁶⁸Ga is of special interest for the production of Ga-radiolabelled metal complexes used as tracer molecules in PET imaging. ⁶⁸Ga is obtained from a ⁶⁸Ge/⁶⁸Ga generator, which means that no cyclotron is required. ⁶⁸Ga decays to 89% by positron emission of 2.92 MeV and its 68 min half life is sufficient to follow many biochemical processes in vivo without unnecessary radiation. With its oxidation state of +III, ⁶⁸Ga forms stable complexes with various types of chelating agents and ⁶⁸Ga tracers have been used for brain, renal, bone, blood pool, lung and tumour imaging.

The short half-life of this nuclide is compatible with quick blood clearance of EGF. The use of derivatives of macrocyclic chelators such as DOTA or NOTA provided stable gallium labeling of somatostatin analogues and oligonucleotides (Hofinann M, Maecke H, Bomer R, et al. Biokinetics and imaging with the somatostatin receptor PET radioligand (68)Ga-DOTATOC: preliminary data. Eur J Nucl Med. 2001; 28(12): 1751-1757; Ugur 0, Kothari P J, Finn R D, et al. Ga-66 labeled somatostatin analogue DOTA-DPhel-Tyr3-octreotide as a potential agent for positron emission tomography imaging and receptor mediated internal radiotherapy of somatostatin receptor positive tumors. Nucl Med Biol. 2002; 29(2): 147-157; Eisenwiener K P, Prata M I, Buschmann I, et al. NODAGATOC, a new chelator-coupled somatostatin analogue labeled with [67/68Ga] and [111In] for SPECT, PET, and targeted therapeutic applications of somatostatin receptor (hsst2) expressing tumors. Bioconjug Chem. 2002; 13(3): 530-541; Froidevaux S, Eberle A N, Christe M, et al. Neuroendocrine tumor targeting: study of novel gallium-labeled somatostatin radiopeptides in a rat pancreatic tumor model. Int J Cancer. 2002; 98(6): 930-937; Velikyan I, Beyer G J, Langstrom B. Microwave-supported preparation of (68)Ga bioconjugates with high specific radioactivity. Bioconjug Chem. 2004; 15(3): 554-560; Velikyan I, Lendvai G, Valila M, et al. Microwave accelerated Ga-68-labelling of oligonucleotides. Journal of Labelled Compounds & Radiopharmaceuticals. 2004; 47(1): 79-89). Earlier experiments with acyclic chelators demonstrated that their attachment to EGF did not reduce affinity of EGF binding to its receptor. For these reasons, coupling of DOTA to EGF might provide an appropriate way for its labeling with gallium. However, there has been no suggestion or teaching in the prior art of how to employ these scientific observations in the imaging of EGFR overexpression in tumors.

Therefore, there is a long-standing need within the medical community for a non-invasive PET tracer for detecting EGFR overexpression in tumors. Such a tracer would be extremely useful in the development of an in vivo non-invasive PET procedure with high sensitivity. Detection of EGFR overexpression in many carcinomas provides important diagnostic information, which can influence patient management. Thus, it is desirable to provide a method for the production of a positron-emitting tracer on the basis of the natural ligand to EGFR, the human recombinant epidermal growth factor (HEGF) and use such a tracer in the imaging of EGFR overexpression in tumors.

Discussion or citation of a reference herein shall not be construed as an admission that such reference is prior art to the present invention.

### SUMMARY OF THE INVENTION

The present invention provides a method for labeling synthesis of radiolabeled gallium complex, comprising:

(a) providing a ⁶⁸Ga³⁺ radioisotope,

(b) reacting said ⁶8Ga³⁺ radioisotope with a chelating agent using microwave activation, and

(c) collecting the resultant radiolabeled gallium complex.

The present invention further provides such radiolabeled gallium complexes as PET tracers. A preferred tracer according to the instant invention is ⁶⁸Ga-DOTA-hEGF.

In yet another embodiment, the invention also provides a method for imaging of EGFR overexpression in tumors comprising administering a radiolabeled gallium complex to a human, wherein the radiolabelled gallium complex is capable of being imaged by positron emission tomography, detecting of EGFR overexpression in tumors by performing positron emission tomography process. In still another embodiment, the invention provides a kit which could be used to obtain ⁶⁸Ga and a kit, which could be used for the production of ⁶⁸Ga-radiolabelled complexes.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 shows specificity of ⁶⁸Ga-DOTA-EGF binding to A431 carcinoma (a) and U343 glioma (b) cell lines. At all time points, EGF receptors on control cells were blocked with a 100-fold excess amount of non-labelled EGF. The binding was specific, since the binding could be suppressed. The presented data are mean values of three measurements and standard deviations.

FIG. 2 shows saturation of ⁶⁸Ga-DOTA-hEGF binding to cultured carcinoma A431 and glioma U343 cells, incubated with different concentrations of ⁶⁸Ga-DOTA-hEGF (0.26-16.9 nM, for A431 cells and 0.14-36 nM for U343 cells) for 2 h on ice in presence or absence of unlabellled HEGF to get non-specific and total binding, respectively. The data was analyzed by GraphPad Prism 3.0. All data points are mean values of at least three data points, and maximal variations are shown.

FIG. 3 shows Internalisation of ⁶⁸Ga-DOTA-EGF after binding to carcinoma A431 and glioma U343 cells. Internalization was determined by acid wash at two different time points. Radioactivity, which was removed from cells by an acidic buffer was considered as membrane-bound, and the rest as internalized. The presented data are mean values of three measurements and standard deviations.

FIG. 4 shows cell-associated ⁶⁸Ga radioactivity as a function of time after interrupted incubation of A431(solid line) and U343(dotted line) with ⁶⁸Ga-DOTA-EGF. The cell associated radioactivity at time zero after the interrupted incubation was considered as 100%. All data points are mean values of three measurements and standard deviations. Both A431 and U343 cell cultures were incubated with ⁶⁸Ga-DOTA-hEGF for 4 h.

FIG. 5(A) is biodistribution of ⁶⁸Ga-DOTA-EGF expressed as % injected dose per gram tissue in tumour bearing nude mice at 30 min time point. FIG. 5(B) shows tumour-to-Organ ratios of ⁶⁸Ga-DOTA-EGF in tumour bearing nude mice at 30 min time point. Mice were intravenously injected with either 0.016 or 0.16 nmol of radiotracer and killed at 30 min time point. Data are presented as mean±SD (n=4).

FIG. 6 Left) is an image showing a summation of frames 20-24 (x-30 min after injection). The tumours can clearly be seen at either side of the head. Right) is a photograph of the positioning of the mouse.

FIG. 7 are pharmacokinetic curves showing the rapid distribution of ⁶⁸Ga-DOTA-EGF (0.16 nmol injected) to liver, kidney and tumours. The excretion into urine is continuous throughout the observation time.

### DETAILED DESCRIPTION OF THE INVENTION

One object of the invention is to provide a method for synthesizing radiolabeled gallium complexes which are useful as radiopharmaceuticals, specifically for use in PET. They are particularly useful for the detection of epidermal growth factor receptor (EGFR) expression in malignant tumors. This is achieved by the method described in the invention.

⁶⁸Ga is obtainable from a ⁶⁸Ge/⁶⁸Ga generator. Such generators are known in the art, see for instance C. Loc'h et al, J. Nucl. Med. 21, 1980, 171-173 or J. Schuhmacher et al. Int. J. appl. Radiat. Isotopes 32, 1981, 31-36. ⁶⁸Ge may be obtained by cyclotron production by irradiation of, for instance Ga₂(SO₄)₃ with 20 MeV protons. It is also commercially available, e.g. as ⁶⁸Ge in 0.5 M HCl. Generally, ⁶⁸Ge is loaded onto a column consisting of organic resin or an inorganic metal oxide like tin dioxide, aluminium dioxide or titanium dioxide. ⁶⁸Ga is eluted from the column with aqueous HCl yielding ⁶⁸GaCl₃. ⁶⁸Ga³⁺ is particularly preferred in the method according to the invention as its production does not require a cyclotron and its 68 min half-life is sufficient to follow many biochemical processes in vivo by PET imaging without long radiation.

Suitable columns for ⁶⁸Ge/⁶⁸Ga generators consist of inorganic oxides like aluminium dioxide, titanium dioxide or tin dioxide or organic resins like resins comprising phenolic hydroxyl groups (U.S. Pat. No. 4,264,468) or pyrogallol (J. Schuhmacher et al., Int. J. appl. Radiat. Isotopes 32, 1981, 31-36). In a preferred embodiment, a ⁶⁸Ge/⁶⁸Ga generator comprising a column comprising titanium dioxide is used in the method according to the invention.

The concentration of the aqueous HC1 used to elute ⁶⁸Ga from the ⁶⁸Ge/⁶⁸Ga generator column depends on the column material. Suitably 0.05 to 5 M HC1 is used for the elution of ⁶⁸Ga. In a preferred embodiment, the eluate is obtained from a ⁶⁸Ge/⁶⁸Ga generator comprising a column comprising titanium dioxide and ⁶⁸Ga is eluted using 0.05 to 0.1 M HCl, preferably about 0.1 M HCl.

In a preferred embodiment of the method according to the invention, a strong anion exchanger comprising HCO₃⁻ as counterions, preferably a strong anion exchanger comprising HCO₃⁻ as counterions, is used. In a further preferred embodiment, this anion exchanger comprises quaternary amine functional groups. In another further preferred embodiment, this anion exchanger is a strong anion exchange resin based on polystyrene-divinylbenzene. In a particularly preferred embodiment, the anion exchanger used in the method according to the invention is a strong anion exchange resin comprising HCO₃⁻ as counterions, quaternary amine functional groups and the resin is based on polystyrene-divinylbenzene.

Suitably, water is used to elute the ⁶⁸Ga from the anion exchanger in the method according to the invention.

The ⁶⁸Ga obtained according to the method of the invention is preferably used for the production of ⁶⁸Ga-radiolabelled complexes, preferably for the production of ⁶⁸Ga-radiolabelled PET tracers that comprise a bifunctional chelating agent, i.e. a chelating agent linked to a targeting vector.

Thus, another aspect of the invention is a method for producing a ⁶⁸Ga-radiolabelled complex by a) obtaining ⁶⁸Ga by contacting the eluate from a ⁶⁸Ge/⁶⁸Ga generator with an anion exchanger comprising HCO₃⁻ as counterions and eluting ⁶⁸Ga³⁺ from said anion exchanger, and

b) reacting the ⁶⁸Ga with a chelating agent.

Preferred chelating agents for use in the method of the invention are those which present ⁶⁸Ga in a physiologically tolerable form. Further preferred chelating agents are those that form complexes with ⁶⁸Ga that are stable for the time needed for diagnostic investigations using the radiolabelled complexes.

Suitable chelating agents are, for instance, polyaminopolyacid chelating agents like DTPA, EDTA, DTPA-BMA, DOA3, DOTA, NOTA, HP-DOA3, TMT or DPDP. Those chelating agents are well known for radiopharmaceuticals and radiodiagnosticals. Their use and synthesis are described in, for example, U.S. Pat. No. 4,647,447, U.S. Pat. No. 5,362,475, U.S. Pat. No. 5,534,241, U.S. Pat. No. 5,358,704, U.S. Pat. No. 5,198,208, U.S. Pat. No. 4,963,344, EP-A-230893, EP-A-130934, EP-A-606683, EP-A-438206, EP-A-434345, WO-A-97/00087, WO-A-96/40274, WO-A-96/30377, WO-A-96/28420, WO-A-96/16678, WO-A-96/11023, WO-A-95/32741, WO-A-95/27705, WO-A-95/26754, WO-A-95/28967, WO-A-95/28392, WO-A-95/24225, WO-A-95/17920, WO-A-95/15319, WO-A-95/09848, WO-A-94/27644, WO-A-94/22368, WO-A-94/08624, WO-A-93/16375, WO-A-93/06868, WO-A-92/11232, WO-A-92/09884, WO-A-92/08707, WO-A-91/15467, WO-A-91/10669, WO-A-91/10645, WO-A-91/07191, WO-A-91/05762, WO-A-90/12050, WO-A-90/03804, WO-A-89/00052, WO-A-89/00557, WO-A-88/01178, WO-A-86/02841 and WO-A-86/02005.

Suitable chelating agents include macrocyclic chelating agents, e.g. porphyrin-like molecules and pentaaza-macrocycles as described by Zhang et al., Inorg. Chem. 37(5), 1998, 956-963, phthalocyanines, crown ethers, e.g. nitrogen crown ethers such as the sepulchrates, cryptates etc., hemin (protoporphyrin IX chloride), heme and chelating agents having a square-planar symmetry.

Macrocyclic chelating agents are preferably used in the method of the invention. In a preferred embodiment, these macrocyclic chelating agents comprise at least one hard donor atom such as oxygen and/or nitrogen like in polyaza- and polyoxomacrocycles. Preferred examples of polyazamacrocyclic chelating agents include DOTA, NOTA, TRITA, TETA and HETA with DOTA being particularly preferred.

Particularly preferred macrocyclic chelating agents comprise functional groups such as carboxyl groups or amine groups which are not essential for coordinating to Ga³⁺ and thus may be used to couple other molecules, e.g. targeting vectors, to the chelating agent. Examples of such macrocyclic chelating agents comprising functional groups are DOTA, NOTA, TRITA or HETA.

In a further preferred embodiment, bifunctional chelating agents are used in the method according to the invention. “Bifunctional chelating agent” in the context of the invention means chelating agents that are linked to a targeting vector. Suitable targeting vectors for bifunctional chelating agents useful in the method according to the invention are chemical or biological moieties, which bind to target sites in a patient's body, when the ⁶⁸Ga-radiolabelled complexes comprising said targeting vectors have been administered to the patient's body. A preferred targeting vector for bifunctional chelating agents useful in the method according to the invention is the natural ligand to EGFR, epidermal growth factor (EGF) or a part, a fragment, a derivative or a complex thereof. The small molecular weight of EGF, 6.2 kDa, enables fast tumour penetration and fast blood clearance, providing good contrast of the image. The use of a positron-emitting label for EGF is particularly advantageous, since PET, compared with SPECT, is a superior detection technique in sensitivity, resolution and quantification. Particularly preferred targeting vector is the human recombinant epidermal growth factor (hEGF) or a part, a fragment, a derivative or a complex thereof.

In a particularly preferred embodiment, macrocyclic bifunctional chelating agents are used in the method according to the invention. Preferred macrocyclic bifunctional chelating agents comprise DOTA, NOTA, TRITA or HETA linked to a targeting vector, preferably to an EGF or a part, a fragment, a derivative or a complex thereof; particularly preferably to an HEGF or a part, a fragment, a derivative or a complex thereof.

The targeting vector can be linked to the chelating agent via a linker group or via a spacer molecule. Examples of linker groups are disulfides, ester or amides, examples of spacer molecules are chain-like molecules, e.g. lysin or hexylamine or short peptide-based spacers. In a preferred embodiment, the linkage between the targeting vector and the chelating agent part of radiolabelled gallium complex is as such that the targeting vector can interact with its target in the body without being blocked or hindered by the presence of the radiolabelled gallium complex.

A preferred aspect of the invention is a method for producing a ⁶⁸Ga-radiolabelled complex by
c) obtaining ⁶⁸Ga by contacting the eluate from a ⁶⁸Ge/⁶⁸Ga generator with an anion exchanger comprising HCO₃⁻ as counterions and eluting ⁶⁸Ga from said anion exchanger, and
d) reacting the ⁶⁸Ga with a chelating agent, wherein the reaction is carried out using microwave activation.

It has been found that the use of microwave activation substantially improves the efficiency and reproducibility of the ⁶⁸Ga-chelating agent complex formation. Due to microwave activation, chemical reaction times could be shortened substantially; i.e. the reaction is completed within 2 min and less. This is a clear improvement as a 10 minutes shortage of the reaction time saves about 10% of the ⁶⁸Ga activity. Furthermore, microwave activation also leads to fewer side reactions and to an increased radiochemical yield, which is due to increased selectivity.

Suitably, a microwave oven, preferably a monomodal microwave oven is used to carry out microwave activation. Suitably microwave activation is carried out at 80 to 120 W, preferably at 90 to 110 W, particularly preferably at about 100 W. Suitable microwave activation times range from 20 s to 2 min, preferably from 30 s to 90 s, particularly preferably from 45 s to 60 s.

A temperature control of the reaction is advisable when temperature sensitive chelating agents, like for instance bifunctional chelating agents comprising peptides or proteins as targeting vectors, are employed in the method according to the invention. Duration of the microwave activation should be adjusted in such a way, that the temperature of the reaction mixture does not lead to the decomposition of the chelating agent and/or the targeting vector. If chelating agents used in the method according to the invention comprise peptides or proteins, higher temperatures applied for a shorter time are generally more favourable than lower temperatures applied for a longer time period.

Microwave activation can be carried out continuously or in several microwave activation cycles during the course of the reaction.

Another aspect of the invention is a kit for obtaining ⁶⁸Ga from a ⁶⁸Ge/⁶⁸Ga generator, which comprises a generator column and a second column that comprises an anion exchanger comprising HCO₃⁻ as counterions.

In a preferred embodiment, the kit further comprises means to couple the columns in series and/or aqueous HCl to elute the ⁶⁸Ga from the generator column and/or water to elute the ⁶⁸Ga from the anion exchanger column. The HCl and the water are preferably aseptically and in a hermetically sealed container.

In another preferred embodiment, the kit according to the invention further comprises a chelating agent, preferably a bifunctional chelating agent, i.e. a chelating agent linked to a targeting vector.

The present invention further provides such radiolabeled gallium complexes as PET tracers. A preferred tracer according to the instant invention is ⁶⁸Ga-DOTA-hEGF.

In yet another embodiment, the invention also provides a method for imaging of EGFR overexpression in tumors comprising administering a radiolabeled gallium complex to a human, wherein the radiolabelled gallium complex is capable of being imaged by positron emission tomography, detecting of EGFR overexpression in tumors by performing positron emission tomography process.

### EXAMPLES

The invention is further described in the following examples which are in no way intended to limit the scope of the invention.

### Example 1

### Chemistry and Radiochemistrv of ⁶⁸Ga-DOTA-hEGF Preparation

I. Materials

Recombinant human epidermal growth factor (hEGF) was purchased from Chemicon (Temecul, Calif., USA). Sodium acetate (99.995%), HEPES (4-(2-Hydroxyethyl) piperazine-1-ethanesulfonic acid), doubly distilled hydrochloric acid (Riedel de Haën) were obtained from Sigma-Aldrich Sweden (Stockholm, Sweden). Sodium dihydrogen phosphate, di-sodium hydrogen phosphate and trifluoroacetic acid (TFA) were obtained from Merck (Darmstadt, Germany). Sulfo-NHS ester of DOTA (1,4,7,10-tetraazacyclododecane-1,4,7,10-tetraacetic acid) was purchased from Macrocyclics (Dallas, Tex., USA). The purchased chemicals were used without further purification. Deionised water (18.2 MΩ), produced with a Purelab Maxima Elga system (Bucks, the UK), was used in all reactions. ⁶⁸Ga was obtained from a ⁶⁸Ge/⁶⁸Ga generator (Cyclotron C., Obninsk, Russia).

II. HPLC Analysis

Analytical liquid chromatography (LC) was performed using a HPLC system from Beckman (Fullerton, Calif., USA) consisting of a 126 pump, a 166 UV detector and a radiation detector coupled in series. Data acquisition and handling was performed using the Beckman System Gold Nouveau Chromatography Software Package. The column used was a Vydac RP 300 Å HPLC column (Vydac, USA) with the dimensions 150 mm×4.6 mm, 5 μm particle size. The applied gradient elution had the following parameters: A=10 mM TFA; B=70% acetonitrile (MeCN), 30% H₂₀, 10 mM TFA with UV-detection at 220 nm; flow was 1.2 mL/min; 0-2 min isocratic 20% B, 20-90% B linear gradient 8 min, 90-20% B linear gradient 2 min. The quantity of ⁶⁸Ga-DOTA-hEGF and radio-impurities retained on the column could be obtained by measuring the activity of the sample injected on the column and the fractions collected from the outlet with a crystal scintillation counter. The overall loss on the system was 10%. The measured activity of the fractions of ⁶⁸Ga-DOTA-hEGF and hydrophilic radio-impurities were in agreement with the respective values obtained from the HPLC chromatograms. The corresponding relative standard deviation values were 7% and 0.5%, respectively for hydrophilic radio-impurities and ⁶⁸Ga-DOTA-hEGF.

III. Preparation of ⁶⁸Ga-DOTA-hEGF

HEGF (32-70 nanomols, 80-180 μL) in 0.08 M borate buffer, pH 9.4, was added to dry N-hydroxy-sulfosuccinimide ester of DOTA (10-20 fold excess) under stirring and the pH was further adjusted to 9.0 by adding borate buffer (240-340 μL). The mixture was left at room temperature for 3-4 hours or overnight. The conjugate was purified on Bio-select RP C18 C-18 SPE column (Vydac). The reaction mixtures was passed slowly though extraction disc, which was then washed with 2 mL of 0.1% TFA. The product was eluted in 1 mL of 70% acetonitrile with 0.1% TFA. The solvent was evaporated using a vacuum centrifuge (Labconco CentriVap Console, Kansas City, Mo., USA), operated at 50° C., and the dry purified product was stored at a temperature below zero.

The labeling of the conjugate was performed using either non-concentrated ⁶⁸Ga-eluate or eluate pre-concentrated, as described previously (Velikyan I, Beyer G J, Langstrom B. Microwave-supported preparation of (68)Ga bioconjugates with high specific radioactivity. Bioconjug Chem. 2004; 15(3): 554-560). In some cases, the eluates from two generators were pre-concentrated in order to increase the amount of ⁶⁸Ga utilized in the labeling reaction. The amount of DOTA-HEGF used in the labeling reaction was 6-10 and 2-5 nanomols, respectively, when using non-concentrated and pre-concentrated ⁶⁸Ga-eluate. Sodium acetate buffer, pH 5.0-5.5, was used for labeling with non-concentrated ⁶⁸Ga, and HEPES buffer, pH 4.6-4.8, was used for pre-concentrated eluate. The labeling was performed by 1 min long microwave heating. The product was purified on Bio-select RP C18 C-18 SPE column as described above. The solvent was then exchanged to PBS (phosphate buffered saline) buffer on NAP-5 columns (Sephadex G-25; Amersham Pharmacia Biotech AB, Uppsala, Sweden). Purity of the conjugate was assessed by HPLC, and concentration of the conjugate and the tracer was determined from UV-HPLC calibration plots.

In order to verify, that binding of ⁶⁸Ga to HEGF was DOTA mediated, a blank experiment was performed. The manipulations were the same as described above, but non-conjugated hEGF was used.

^{69,71}Ga of natural isotope composition was complexed to DOTA-HEGF using the same protocol. ^{69,71}Ga-DOTA-hEGF characterized with LC-ESI-MS was used for the identification of the radio-HPLC chromatogram signals.

IV. Microwave Heating and LC-ESI-MS Analysis

The microwave heating was performed in a SmithCreator™ monomodal microwave cavity producing continuous irradiation at 2450 MHz (Personal Chemistry AB, Uppsala, Sweden). The temperature, pressure and irradiation power were monitored during the course of the reaction. The reaction vial was cooled down with pressurized air after completed irradiation.

Liquid chromatography electrospray ionization mass spectrometry (LC-ESI-MS) was performed using the Waters Micromass Quattro Premier Mass Spectrometer (Micromass, UK) and an HPLC system from Alliance (Waters 269, UK) with Photodiode Array UV detector. The column used was an Antlantis, dC 18, RP HPLC column with the dimensions 100 mm×2.1 mm, 3 μm particle size. Isocratic elution was applied with the following parameters: A=10 mM Formic acid; B=100% acetonitrile (MeCN), with UV-detection at 210-400 nm; flow was 0.3 mL/min. LC-ESI-MS was performed with positive mode scanning and selected ion recording (SIR) detecting [M+6H]⁶⁺, [M+7H]⁷⁺ and [M+8H]⁸⁺ species. HEGF was detected at m/z=781.5 for [M+8H]⁸⁺, m/z=893 for [M+7H]⁷⁺ and m/z=1042 for [M+6H]⁶⁺. Reconstitution of the data gave M=6244.67±1.15. (DOTA)₁-hEGF was detected at m/z=829.75 for [M+8H]⁸⁺, m/z=948.13 for [M+7H]⁷⁺ and m/z=1105 for [M+6H]⁶⁺. Reconstitution of the data gave M=6629.95±0.05. (DOTA)₂-hEGF was detected at m/z=878 for [M+8H]⁸⁺, m/z=1003.3 for [M+7H]⁷⁺ and m/z=1170.36 for [M+6H]⁶⁺. Reconstitution of the data gave M=7016±0.08. (DOTA)₃-hEGF was detected at m/z=926.29 for [M+8H]⁸⁺, m/z=1058.47 for [M+7H]⁷⁺ and m/z=1234.72 for [M+6H]⁶⁺. Reconstitution of the data gave M=7402±0.1. (Ga-DOTA)₁-hEGF was detected at m/z=838.5 for [M+8H]g+, m/z=958.13 for [M+7H]⁷⁺ and m/z=1117.66 for [M+6H]⁶⁺. Reconstitution of the data gave M=6699.95±0.05. (Ga-DOTA)₂-hEGF was detected at m/z=896.05 for [M+8H]⁸⁺, m/z=1023.3 for [M+7H]⁷⁺ and m/z=1193.69 for [M+6H]⁶⁺. Reconstitution of the data gave M=7157.55±2.47. (Ga-DOTA)₃-hEGF was detected at m/z=952.54 for [M+8H]8+, M/Z=1088.47 for [M+7H]⁷⁺ and m/z=1269.72 for [M+6H]⁶⁺. Reconstitution of the data gave M=7612.31±0.05.

V. Results

⁶⁸Ga-DOTA-HEGF was synthesized by a two-step procedure where HEGF was initially conjugated to a bifunctional chelator, DOTA, and thereafter labeled with ⁶⁸Ga via a complexation reaction of ⁶⁸Ga with the chelator. In the conjugation step, the one of carboxylic groups of the DOTA chelate was coupled to an amine functionality of the peptide forming an amide bond (Scheme 1). The basic pH required for the conjugation reaction was provided by borate buffer. HEGF contains one terminal and two lysine amino groups. Consequently, the conjugation reaction of hEGF resulted in the formation of a mixture of molecules with one, two and three DOTA fragments, as determined by LC-ESI-MS analysis.

The microwave-accelerated labeling of the conjugates (Scheme 1) was performed using a non-concentrated or a pre-concentrated generator ⁶⁸Ga-eluate. The labeling yield was 60±10% (N=3) in the case of non-concentrated conjugate. The use of pre-concentration enabled to increase 77±4% (N=3). Pre-concentration of eluate allowed to obtain specific radioactivity of 28 MBq/nmol. Attachment of ⁶⁸Ga to HEGF was DOTA-mediated, since the same treatment of non-conjugated HEGF din not provide any labeled peptide. The radiochemical purity of the tracers in the study exceeded 99%. The tracer proved to be stable in the PBS, with no additional radio-HPLC signals during the stability assay of four hours.

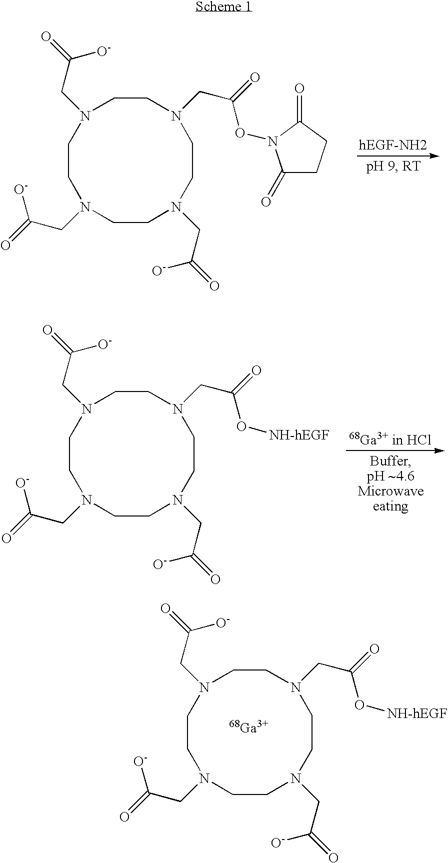

### Example 2

### Cell Binding and Retention Experiments

I. Cell Culture

The human squamous carcinoma cell line A431 (ATCC, CLR 1555, Rocksville, Md., USA) and the malignant glioma cell line U343MGaC12:6 (Westermark B, Magnusson A, Heldin C H. Effect of epidermal growth factor on membrane motility and cell locomotion in cultures of human clonal glioma cells. J Neurosci Res. 1982; 8(2-3): 491-507) (from now on denoted U343) were used in all cell experiments. This A431 cell line is reported to express approximately 2×10⁶ EGFR per cell, and the U343 cell line express approximately 5.5×10⁵ EGFR per cell. The cells were cultured in Ham's F10 medium (Biochrom Kg), supplemented with 10% fetal calf serum (Sigma), L-glutamine (2 mM) and PEST (penicillin 100 IU/ml and streptomycin 100 μg/ml) both from Biochrom Kg. During cell culture and cell experiments (unless otherwise stated) cells were grown at 37° C. in incubators with humidified air, equilibrated with 5% CO₂. The cells were trypsinized with trypsin-EDTA (0.25% trypsin, 0.02% EDTA in PBS without Ca and Mg) from Biochrom Kg.

II. Binding of ⁶⁸Ga-DOTA-EGF to the Cells

A431 and U343 cells were cultured in 3 cm Petri dishes (approximately 3.5×10⁵ and 1.9×10⁵ cells per dish, respectively). After washing the cells once, ⁶⁸Ga-DOTA-EGF in cell culture medium (35 ng/dish, 50 kBq/dish for A431 cells and 5 ng/dish, 20 kBq/dish for U343 cells) was added. The concentration of the added tracer was 0.26-16.9 nM, for A431 cells and 0.14-36 nM for U343 cells. To some dishes, a molar excess of EGF (5 or 3 μg/dish) was added together with the labelled conjugate, in order to estimate the binding specificity of the ⁶⁸Ga-DOTA-EGF conjugate. After 0.5-6 h incubation at 37° C., the cells were washed six times with cold serum free medium, and they were then harvested using 0.5 ml trypsin-EDTA (15 min, 37° C.). The trypsination was terminated with addition of 1 ml cell culture medium, and part of the cell suspension (0.5 ml) was used for cell counting while the rest was measured in a gamma counter.

In order to estimate the cellular internalization of the ⁶⁸Ga-DOTA-EGF conjugate, a number of additional cell dishes were used during the binding study to separate the membrane bound fraction of the conjugate from internalized radioactivity. Instead of trypsinising the cells, treatment with 0.5 ml ice-cold 0.1 M glycin-HCl buffer, pH 2.5 for 6 min at 0° C. was used to extract the membrane bound fraction of the conjugate. An additional 0.5 ml of the glycin-HCl buffer was used to wash the cells once. The remaining radioactivity, considered to be internalized radioactivity, was collected by treatment with 0.5 ml 1 M NaOH solution at 37° C. for about 60 min. Another 0.5 ml NaOH solution was used for washing. The collected fractions were measured in an automated gamma counter.

The binding of ⁶⁸Ga-DOTA-EGF to A431 cells and U343 cells on ice was also studied, in order to determine the time required for binding in the saturation study. Cell dishes placed on ice were incubated with ice cold ⁶⁸Ga-DOTA-EGF solution for 0.5-4 h. The cells were then washed, trypsinized and counted as described above.

III. Cellular Retention of Radioactivity, Saturation Assay and Animal Tumor Model

The cellular retention of radioactivity was studied after 1 h of incubation with ⁶⁸Ga-DOTA-EGF. After the incubation, the cells were washed thoroughly to eliminate unbound conjugate, and the incubation was then continued in fresh cell culture medium. After 0.5-4 h, the cells were trypsinized, counted and measured for radioactivity, as described above.

The equilibrium dissociation constant, K_{d}, was determined from a saturation study with ⁶⁸Ga-DOTA-EGF on A431 cells and U343 cells. Cells cultured in 24-well dishes (approximately 3.1×10⁴ A431 cells per well and 7.8×10⁴U343 cells per well) were placed on ice, and ice cold ⁶⁸Ga-DOTA-EGF solutions of different concentrations (0.26-16.9 nM for A431 and 0.14-36-nM for U343) were added. For each concentration, the unspecific background binding was studied by adding a 100 times excess of unlabelled EGF to some wells. After 2 h of incubation (the time was determined from the results of the uptake study on ice), the cells were washed six times with cold serum free medium. The cells were then trypsinized with 0.5 ml of trypsin-EDTA (15 min at 37° C.), and the cells were counted and measured for radioactivity in a gamma counter.

The in vivo studies were carried out in adult female Balb/c nu/nu mice (21-25 g) (Mollegard, Denmark) with tumor xenografts. All animals were handled according to the guidelines by the Swedish Animal Welfare Agency, and the experiments were approved by the local Ethics Committee for Animal Research. The mice were injected subcutaneously with A431 tumor cells (approximately 7 million cells per tumor in 100 μl cell culture medium) in both front legs. The tumors were allowed to grow for 12-13 days before the experiments were performed, and had then reached a weight of 0.1-0.8 g.

IV. Results

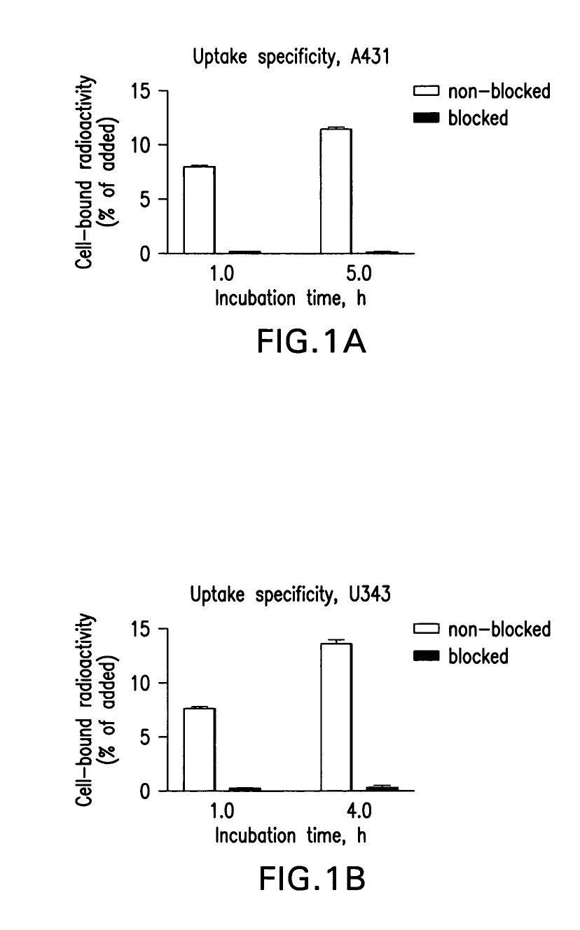

The binding specificity of ⁶⁸Ga-DOTA-EGF to EGFR-expressing cell lines in vitro is shown in FIG. 1. Cervical carcinoma A431 and glioma U343 cell lines, which have a documented expression of EGFR, were used in the cell tests. In order to demonstrate that binding is receptor-specific, a large amount of non-labelled EGF was added to cells in the control experiments, in order to saturate EGFR. Results of the binding specificity experiments demonstrated that the binding of ⁶⁸Ga-DOTA-EGF to both cell lines might be prevented by receptor saturation at all tested data points. This indicates that binding of the labelled conjugate is receptor specific.

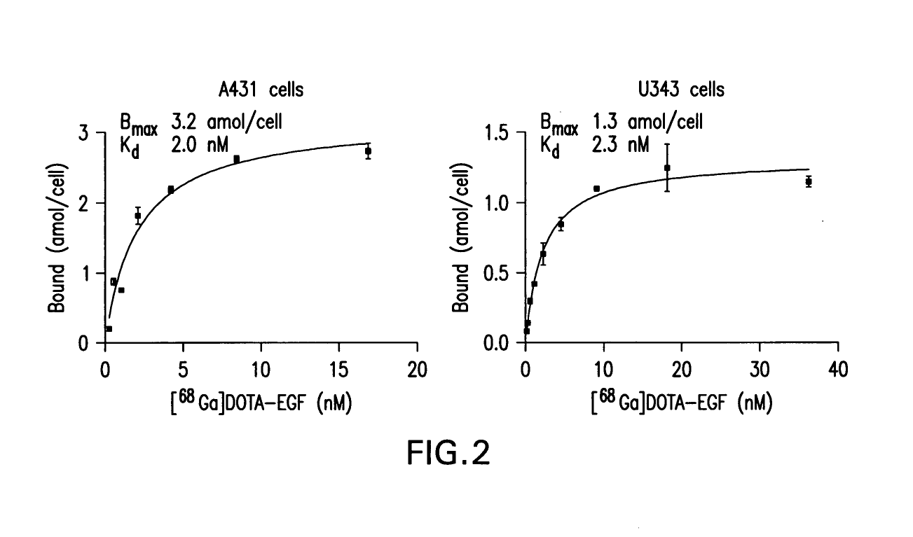

The results of the saturation experiments with ⁶⁸Ga-DOTA-EGF on cervical carcinoma A431 and glioma U343 cell lines are shown in FIGS. 2A and 2B, respectively. The specific binding in amol/cell is plotted against the total molar concentration of added radiolabelled conjugate, and the result is analyzed by nonlinear regression using the GraphPad Prism Software. Both curves seem to have reached a maximum value, indicating saturation. The obtained Kd values were in an excellent agreement, 2.0 nM for A431 and 2.3 nM for U343 cells. The maximum amount of binding sites per cell, 7.8×10⁵ for U343 cells corresponds reasonably well with 5.4×10⁵ as previously determined for a [¹¹¹In]-Bz-DTPA-EGF conjugate (22). The number of binding sites for A431, 1.9 millions per cell is also in good agreement with literature data.

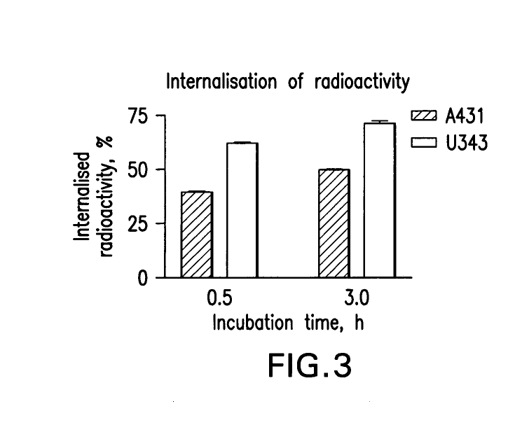

In this study, the degree of internalization was estimated by acid wash. Radioactivity, which was removed from cells by an acidic buffer was considered as membrane-bound, and the rest as internalized. Results of such experiments are shown in FIG. 3 which shows that internalization of ⁶⁸Ga-DOTA-EGF is a rapid process in both cell lines. However, results of these experiments indicate that the internalization rate was faster in glioma U343 cells as compared to A431. This may possibly be due to the documented capacity of A431 cells to recycle internalized receptors to the cell surface. More than 50% of the radioactivity was internalized at 30 min after the start of incubation in the case of glioma U343 cells.

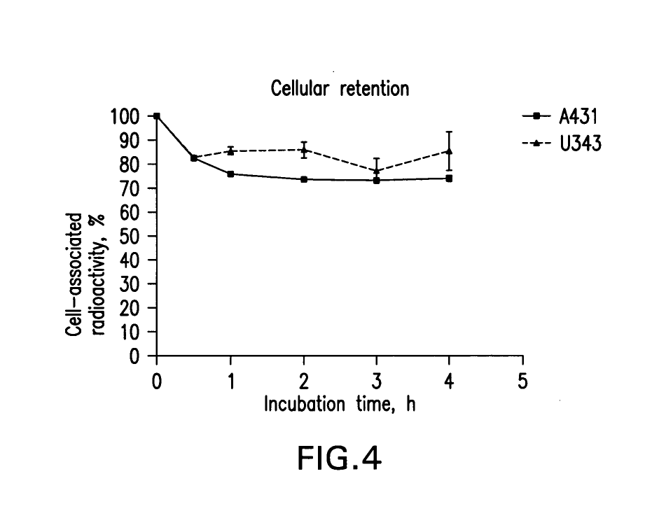

The retention pattern of radioactivity after interrupted incubation with ⁶⁸Ga-DOTA-EGF for A431 and U343 cells was similar for both cell lines (FIG. 4). An initial drop of radioactivity, which was most likely due to dissociation of membrane-bound conjugates, was followed by a relatively constant amount of cell-bound ⁶⁸Ga. Both cell-lines demonstrated good retention, when more than 70% of the radioactivity was still cell-associated 4 hours, more than 3 half-lives of the labell, after interrupted incubation.

### Example 3

### Biodistribution Studies

I. Biodistribution in Mice with A431 Tumor Xenografts

In order to estimate an influence of amount injected conjugate on uptake in tumors and normal tissues, a biodistribution study was performed. Mice with A431 tumor xenografts were injected intravenously with 50 μl ⁶⁸Ga-DOTA-EGF solution (0.16 nmol or 0.016 nmol in PBS per animal), and 30 min post injection the animals were sacrificed and dissected. The mice were anesthetised by an intraperitoneal injection of a mixture of Rompun (1 mg/ml) and Ketalar (10 mg/ml), 0.2 ml per 10 g of animal weight, and killed by heart puncture. In addition to the tumors, blood, heart, pancreas, spleen, stomach, liver, kidneys, lungs, small and large intestine, muscle, bone and salivary gland were collected, weighed and measured in an automated gamma counter. The tails were also measured for radioactive content, in order to determine the accuracy of the injections. Organ values were calculated as percent of injected activity per g of organ (% IA/g).

II. Results

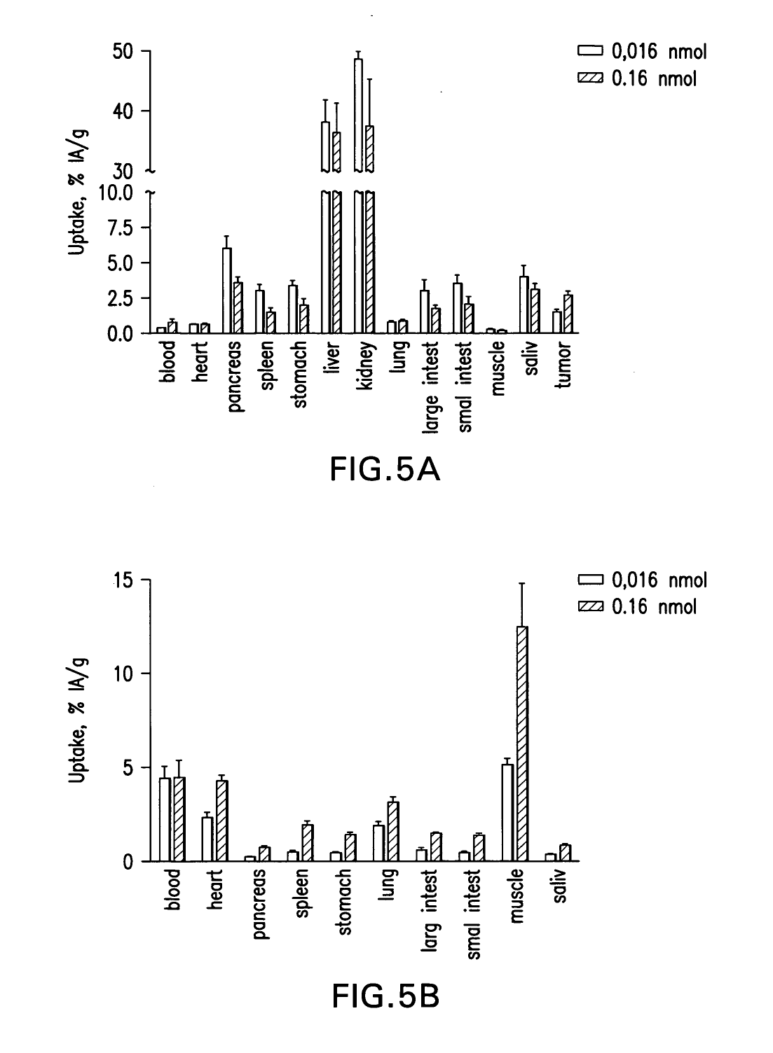

A summary of the biodistribution data for ⁶⁸Ga-DOTA-EGF in A431 tumour-bearing mice is shown in FIG. 5. The measurement of the organ radioactivity 30 min after i.v. administration of ⁶⁸Ga-DOTA-EGF showed the highest values in the kidneys and liver for both conjugates. The lower level of radioactivity accumulation was observed in pancreas, salivary gland, small and large intestine, stomach and spleen. The uptake of ⁶⁸Ga-DOTA-EGF in the A431 tumour xenografte was 1.51±0.16% IA/g and 2.69±0.29% IA/g, for 0.016 and 0.16 nmol of injected conjugate respectively (p=0.036). The radiotracer had a rapid blood clearance, with less than 1% IA/g remaining in the circulation at 30 min time point for both conjugates (no significant difference). There were statistically significant decrease of the radioactivity uptake in pancreas, spleen and stomach, when larger amount of conjugate was injected. Influence of increased amount of conjugate was even more pronounced, when tumor-to-normal organs were considered. Though, there were no difference in tumor-to-blood ratio, 4.42±1.81% IA/g and 4.50±2.53% IA/g, for 0.016 and 0.16 nmol of injected conjugate respectively (p=0.036), there were statistically significant increase of tumor-to-organ ratios for heart, pancreas, stomach, spleen, lungs, intestines, muscles and salivary glands in he case when 0.16 nmol of conjugate was injected.

### Example 4

### MicroPET Imaging

Imaging was performed on a microPET R4 scanner (Concorde Microsystems, Inc.), with a computer-controlled bed and 10 cm transaxial and 8 cm axial field of view (FOV). It operates exclusively in 3-dimensional list mode and has no septa. All raw data were first sorted into 3-dimensional sinograms, followed by Fourier rebinning and 2-dimensional filtered back projection image reconstruction resulting in images with 2 mm resolution. The mice were taken to the laboratory just before the experiment. After a short period of heating under a red-light bulb the animal was placed in a cylinder connected to an isoflurane vaporizer adjusted to deliver 2% isoflurane in a 45/55% mixture of oxygen and air. When the animal was unconscious a heparinised venous catheter was placed in a tail vein and connected to a 1 ml syringe with 0.9% NaCl and 10 IU heparin. The animal was subsequently placed on the camera bed with its abdomen down and forelegs with tumours stretched out forward as much as possible from the body and covered with saran wrap to minimize heat and water loss. Heated air (40° C.) was blown on the animal to reduce the loss of body temperature during the experiment. The tracer was injected as a bolus dose shortly after the camera start in a volume of 100 μl followed by 100 μl saline. After completion of the study the animals were decapitated under anesthesia and blood, liver, and kidney samples were collected for radioactivity measurements.

Scatter correction, random counts and dead time correction were all incorporated into the reconstruction algorithm. Radiation attenuation in each animal was measured with two rotating rod sources containing ⁶⁸Ge/⁶⁸Ga before tracer injection and the images were corrected for radiation attenuation. All PET studies started with a 20 min transmission scan. The amount of the injected activity was 2.0±0.5 MBq. Two different imaging protocols were employed in this study. The acquisition times were as follows: Protocol 1 (duration 120 min) 10×30 s, 5×120 s, and 5×300 s, 8×600 s; Protocol 2 (duration 30 min) 10×30 s, 5×60 s, 10×120 s. Regions of interest (ROIs) were drawn on liver, kidney, bladder, salivary gland and tumours. Pharmacokinetic curves, representing the radioactivity concentrations (percentage of injected dose per gram of tissue), versus time after injection were determined accordingly. The uptake index was calculated as activity in organ [kBq/mL]/injected dose [kBq]×100%.

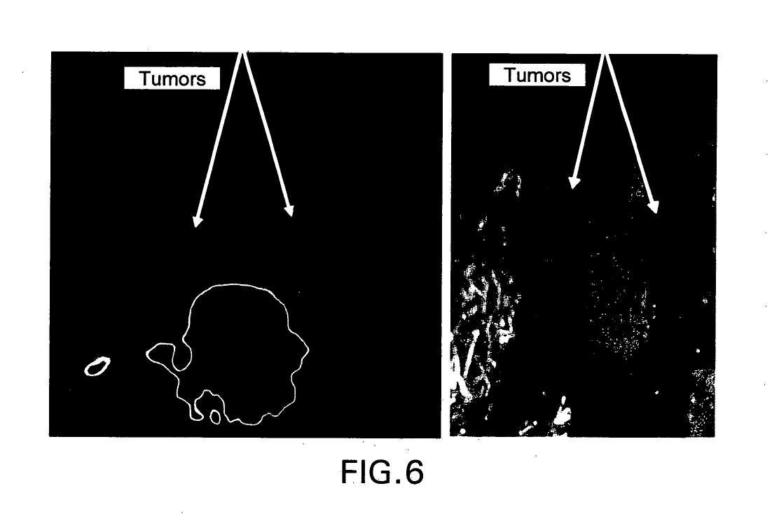

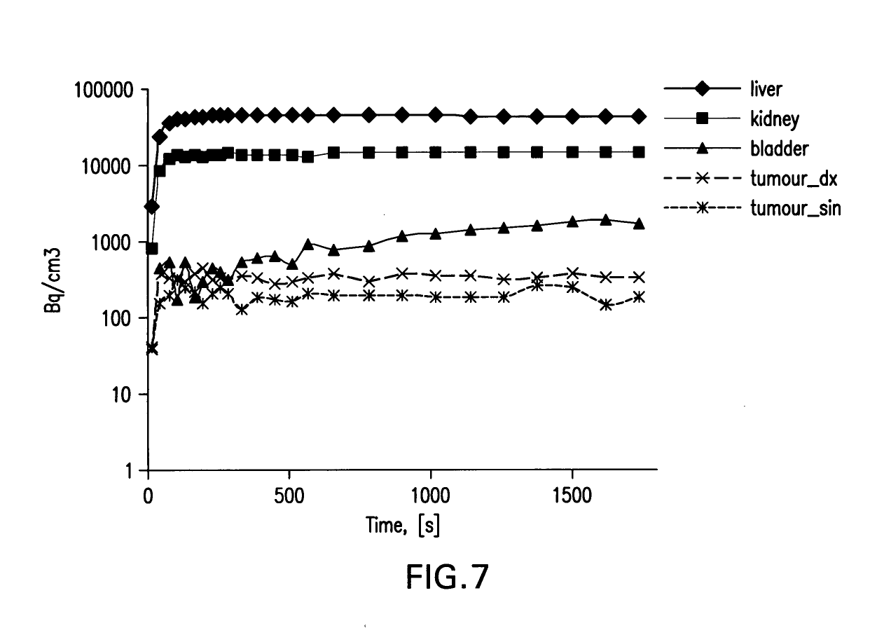

The localization of ⁶⁸Ga-DOTA-EGF in tumor-bearing mice as determined by microPET imaging (FIG. 6) was followed by activity measurements of blood, liver, and kidney samples collected after decapitation of the animal. The image of a tumor bearing mouse 30 min after administration of 2.0 MBq (with specific radioactivity of 12-20 MBq/nmol) ⁶⁸Ga-DOTA-EGF is shown to the left of FIG. 6. The evaluation results of the microPET image are correlated with the activity measurements of blood, liver, and kidney samples. Both right and left leg tumors were visible with clear contrast from the adjacent background. Prominent uptake was observed in the liver and kidneys, and clearance of the activity through the urinary bladder was evident (FIG. 7). The distribution to tumors and salivary gland were slower. Uptake data derived from microPET and biodistribution studies were found to be in agreement and compared with data obtained from the post imaging tissue sampling.

### SPECIFIC EMBODIMENTS, CITATION OF REFERENCES

The present invention is not to be limited in scope by specific embodiments described herein. Indeed, various modifications of the inventions in addition to those described herein will become apparent to these skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for labeling synthesis of radiolabeled gallium complex, comprising:
(a) providing a ⁶⁸Ga³⁺ radioisotope,
(b) reacting said ⁶⁸Ga³⁺ radioisotope with a chelating agent using microwave activation, and (c) collecting the resultant radiolabeled gallium complex.

2. A method of claim 1, wherein the chelating agent is a macrocyclic chelating agent.

3. A method of claim 1, wherein the chelating agent comprises hard donor atoms, preferably O and N atoms.

4. A method of claim 1, wherein the chelating agent is a bifunctional chelating agent.

5. A method of claim 1, wherein the chelating agent is a bifunctional chelating agent comprising a targeting vector.

6. A method of claim 5, wherein the targeting vector is an EGF, or a part, a fragment, a derivative or complex thereof.

7. A method of claim 6, wherein the EGF is an hEGF.

8. A method of claim 1, wherein the microwave activation is carried out at 80 to 120 W, preferably at 90 to 110 W.

9. A method of claim 1, wherein the microwave activation is carried out for 20 seconds to 2 minutes, preferably for 30 seconds to 90 seconds.

10. A method of claim 1, wherein the ⁶⁸Ga³⁺ is obtained by contacting the eluate from a ⁶⁸Ge/⁶⁸Ga generator with an anion exchanger and eluting ⁶⁸Ga³⁺ from said anion exchanger.

11. A method of claim 10, wherein the ⁶⁸Ge/⁶⁸Ga generator comprises a column comprising titanium dioxide.

12. A method of claim 11, wherein the anion exchanger comprises HCO₃⁻ as counterions.

13. A method of claim 10, wherein the anion exchanger is a strong anion exchanger.

14. A radiolabeled gallium complex synthesized according to a method of claim 1.

15. The radiolabeled gallium complex of claim 14 with the formula of ⁶⁸Ga-DOTA-HEGF.

16. A method for imaging of EGFR overexpression in tumors comprising administering a radiolabeled gallium complex to human, wherein the radiolabeled gallium complex is capable of being imaged by PET, detecting EGFR overexpression in tumors by performing PET.

17. A method of claim 16, wherein the radiolabeled gallium complex is ⁶⁸Ga-DOTA-hEGF.
